(19) United States
(12) Patent Application Publication
(10) Pub. No.: US 2021/0387973 A1
(43) Pub. Date: Dec. 16, 2021

(54) SUBSTITUTED HETEROCYCLIC COMPOUNDS AND THEIR USE AS RETINOID-RELATED ORPHAN RECEPTOR (ROR) GAMMA-T INHIBITORS

(71) Applicant: Technische Universiteit Eindhoven, Eindhoven (NL)

(72) Inventors: Lucas BRUNSVELD, Eindhoven (NL); Richard Gerard DOVESTON, Eindhoven (NL); Seppe Frans Roman LEYSEN, Eindhoven (NL); Lech-Gustav MILROY, Eindhoven (NL); Femke Anouk MEIJER, Eindhoven (NL); Marcel SCHEEPSTRA, Eindhoven (NL); Christian OTTMANN, Eindhoven (NL)

(21) Appl. No.: 17/422,645

(22) PCT Filed: Jan. 15, 2020
(86) PCT No.: PCT/NL2020/050020

§ 371 Date: Jul. 13, 2021

(30) Foreign Application Priority Data
Jan. 15, 2019 (NL) 2022404

(51) Int. Cl. C07D 413/04; C07D 261/08

(52) U.S. Cl. CPC C07D 413/04; C07D 261/08

(57) ABSTRACT

The present invention relates to the field of (auto)immune diseases. The present invention provides heterocyclic compounds having an ROR-yt inhibitory action. It also provides for the use of the compounds as a medicament, in particular for use in the treatment of (auto)immune diseases. Also provided are pharmaceutical compositions comprising the compounds having ROR-yt inhibitory action.

### FIELD OF THE INVENTION

The present disclosure relates to the field of (auto)immune diseases and/or allergic disorders. The present disclosure provides heterocyclic compounds having an ROR-γt inhibitory action. It also provides for the use of these compounds as a medicament, in particular for use in the treatment of, for example, (auto)immune diseases and/or allergic disorders. Also provided are pharmaceutical compositions comprising compounds having ROR-γt inhibitory action.

### BACKGROUND OF THE INVENTION

The retinoic acid receptor-related orphan nuclear receptor (ROR) and its isoform ROR-γt (herein collectively referred to as “ROR-γt”) are transcription factors that belong to the steroid hormone receptor superfamily (Jetten 2006. Adv. Dev Biol. 16: 313-355.).

ROR-γt is required for the differentiation of T cells and secretion of Interleukin 17 (IL-17) from a subset of T cells named Th17 cells (Ivanov, Cell 2006, 126, 1121-1133). These cells have been proposed to protect the host from infection by secreting inflammatory cytokines such as IL-17 (also called IL-17A), IL-17F, IL-22, and TNFα. These cytokines are essential in regulating various immune responses, such as inflammatory responses to antigens.

Th17 cells also fulfil important roles in activating and directing immune responses in various (auto)immune diseases, including but not limited to experimental autoimmune encephalomyelitis (EAE), collagen-induced arthritis (CIA), inflammatory bowel disease (IBD), and graft-versus-host disease. The cells have also been implicated in other conditions including asthma, psoriasis, rheumatoid arthritis, multiple sclerosis, and Crohn's disease.

Indeed the rationale for the use of a ROR-γt targeted therapy for the treatment of inflammatory diseases is based on the emerging evidence that Th17 cells and the cytokine IL-17 contribute to the initiation and progression of the pathogenesis of several autoimmune diseases and/or allergic disorders including psoriasis, rheumatoid arthritis, systemic lupus erythromatosis, scleroderma, Type II diabetes, asthma, allergic rhinitis, allergic eczema, multiple sclerosis, juvenile rheumatoid arthritis, juvenile idiopathic arthritis, inflammatory bowel diseases, ulcerative colitis, Crohn's disease, graft versus host disease, spondyloarthropathies and uveitis (See, for example, Miossec, Nature Drug Discovery 2012, 11, 763-776) -536). For example, studies with neutralizing antibodies to IL-17 and its receptor IL-17RA (Leonardi 2012, New England Journal of Medicine, 366, 1190-1199; Papp 2012, New England Journal of Medicine 366, 1181-1189) in psoriasis highlight the role of IL-17 in the pathogenesis of this disease. As such, attenuation of IL-17 secretion from the activated Th17 T cells via inhibition of ROR-γt may offer similar therapeutic benefit. Therefore, a medicament that inhibits the action of ROR-γt is expected to show a treatment effect on various immune disease and/or allergic disorders.

Existing therapeutic drugs in the indicated diseases show only limited effects, and therefore, the availability of novel therapeutic drugs is desired. In light of this, compounds, compositions, methods and uses for the prophylaxis and/or for the treatment of (auto)immune diseases and/or allergic reactions would be highly desirable. Accordingly, the technical problem underlying the present invention can been seen in the provision of such compounds, compositions, methods and uses for complying with any of the aforementioned needs. More in particular the present invention aims to provide compounds having ROR-γt inhibitory action, and that are useful for the prophylaxis and/or treatment of (auto)immune diseases and/or allergic reaction including psoriasis, rheumatoid arthritis, systemic lupus erythromatosis, scleroderma, Type II diabetes, asthma, allergic rhinitis, allergic eczema, multiple sclerosis, juvenile rheumatoid arthritis, juvenile idiopathic arthritis, inflammatory bowel diseases, ulcerative colitis, Crohn's disease, graft versus host disease, spondyloarthropathies and uveitis and the like.

The technical problem is solved by the embodiments characterized in the claims and/or described herein below.

### SUMMARY OF THE INVENTION

In a first aspect the application relates to a compound having formula (I):

(I)

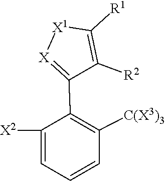

or salts or solvates thereof;

wherein:
X and X¹ are independently N or O;
X² and X³ are each independently an H or a halogen, wherein preferably X² is Cl and X³ is F;
R¹ is a hydrocarbyl, preferably alkyl or aryl, wherein said alkyl and aryl may be unsubstituted or substituted, wherein said aryl and alkyl may comprise one or more heteroatoms, wherein said alkyl may be linear, branched or cyclic; and
R² is

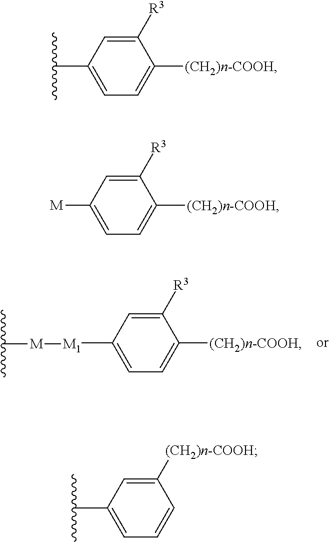

wherein
R³ is a halogen or H, wherein preferably the halogen is F;
n is 0 or an integer equal or greater than 1; and
M and M₁ are, each independently selected from the group consisting of —NH—, —CH₂—, —SO₂, —NH—CH₂—, —COH, —O—, ═N—, and —C(O).

In a second aspect, the invention relates to a composition comprising a compound represented by formula (I) according to the first aspect of the invention, or a salt or solvate thereof, and preferably at least one carrier.

In a third aspect, the invention relates to a pharmaceutical composition comprising a compound represented by formula (I) according to the first aspect of the invention, or a pharmaceutically acceptable salt or solvate thereof, and preferably at least one pharmaceutically acceptable carrier.

In a fourth aspect, the invention relates to a compound represented by formula (I) according to the first aspect of the invention, or a pharmaceutically acceptable salt or solvate thereof, for use as a medicament.

In a fifth aspect, the invention relates to a compound represented by formula (I) according to the first aspect of the invention, or a pharmaceutically acceptable salt or solvate thereof, for use in the prophylaxis or treatment of an autoimmune disease or allergic disorder in a subject. In an embodiment, the autoimmune disease or allergic disorder is selected from psoriasis, rheumatoid arthritis, systemic lupus erythromatosis, scleroderma, Type II diabetes, asthma, allergic rhinitis, allergic eczema, multiple sclerosis, juvenile rheumatoid arthritis, juvenile idiopathic arthritis, inflammatory bowel diseases, ulcerative colitis, Crohn's disease, graft versus host disease, spondyloarthropathies and uveitis.

In a sixth aspect, the invention relates to a compound represented by formula (I) according to the first aspect of the invention, or a pharmaceutically acceptable salt or solvate thereof, for use in a method for the prophylaxis or treatment of an autoimmune disease or allergic disorder in a subject wherein the method comprises administering to the subject a therapeutically effective amount of a compound represented by formula (I) according to the first aspect of the invention, or a pharmaceutically acceptable salt or solvate thereof.

In a seventh aspect, the invention relates to a compound represented by formula (I) according to the first aspect of the invention, or a pharmaceutically acceptable salt or solvate thereof, for use in a method for inhibiting ROR-yt in a subject wherein the method comprises administering to the subject a compound represented by formula (I) according to the first aspect of the invention, or a (pharmaceutically) acceptable salt or solvate thereof.

In an eight aspect, the invention relates to a use of a compound represented by formula (I) according to the first aspect of the invention, or a (pharmaceutically) acceptable salt or solvate thereof, for the production of a pharmaceutical composition.

In a ninth aspect, the invention relates to a use of a compound represented by formula (I) according to the first aspect of the invention, or a (pharmaceutically) acceptable salt or solvate thereof as an inhibitor of ROR-yt.

Definitions

The section headings used herein are for organizational purposes only and are not to be construed as limiting the subject matter described.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

The term “half maximal inhibitory concentration (IC₅₀)” as used in the present application means the concentration of a particular inhibitor needed to inhibit a given activity by a half.

In the present application the IC₅₀ is determined according to a biochemical TR-FRET assay. These assays are conducted using 100 nM N-terminal biotinylated SRC-1 box2 peptide (Biotin-N-PSSHSSLTARHKILHRLLQEGSPSD-CONH₂) and 20 nM His₆-RORγt-LBD in buffer containing 10 mM HEPES, 150 mM NaCl, 5 mM DTT, 0.1% BSA (w/v) and 0.1 mM CHAPs, pH 7.5. A terbium labelled anti-His antibody (CisBio Bioassays, 61HISTLA) and D2-labelled streptavidin (CisBio Bioassays, 610SADLA) are used at the concentrations recommended by the supplier. Compounds (dissolved in DMSO) are titrated using a 2× dilution series in Corning white low volume, low binding, 384-well plates at a final volume of 10 μL The final DMSO concentration being 2% v/v throughout. The plate is incubated at a temperature of between 15° C. and 25° C., preferably 20° C., for 30 min and centrifuged before reading (excitation=340 nm; emission=665 nm and 620 nm) on a Tecan infinite F500 plate reader using the parameters recommended by CisBio Bioassays. The data is analyzed with Origin Software. The dose-response curve was fitted represented by:

$$y = {A}_{1} + \frac{{A}_{1} - {A}_{2}}{1 + 1 {0}^{( log ( {x}_{0} ) - x ) p}}$$

Where y is the FRET ratio, A₁ is the bottom asymptote, A₂ is the top asymptote, p is the Hills slope and x is the ligand concentration. Where dose-response curves do not reach a bottom asymptote this is fixed at the value of the negative control.

The term “halogen” as used in the present application means any of the elements fluorine, chlorine, bromine, iodine, and astatine, occupying group VIIA (17) of the periodic table of elements.

The term “aryl” as used in the present application means a functional group or substituent derived from an aromatic mono- and poly-carboxyclic ring systems in which the individual carbocyclic rings in the polyrings systems are fused or attached to each other via a single bond. Examples: phenyl and napthyl. An aryl can be unsubstituted or substituted.

The term “substituted aryl” as used in the present application means an aryl group substituted with one or more functional groups which is/are attached to the aryl group. For example, functional groups, such as, hydroxyl, bromo, fluoro, chloro, iodo, mercapto or thio, cyano, alkylthio; heterocyclyl, carboxyl, carbalkoyl, alkyl, alkenyl, nitro, amino, alkoxyl, amido, and the like. An example of a substituted aryl is phenoxyl.

The term “cyclic alkyl comprising one or more heteroatoms” as used in the present application means an alkyl of a stable 4- to 8-membered, saturated or unsaturated monocyclic ring; wherein the monocyclic ring contains one or more heteroatoms selected from N, O and S and a balance of carbon atoms. The saturated or unsaturated monocyclic ring can be unsubstituted or substituted. Examples of cyclic alkyl comprising one or more heteroatoms are pyrrolyl, furyl, and thienyl.

The term “substituted cyclic alkyl comprising one or more heteroatoms” as used in the present application means a cyclic alkyl comprising one or more heteroatoms, which is substituted with one or more functional groups which is/are attached to the cyclic alkyl comprising one or more heteroatoms. For example, functional groups, such as, hydroxyl, bromo, fluoro, chloro, iodo, mercapto or thio, cyano, alkylthio; aryl, heteroaryl, carboxyl, carbalkoyl, alkyl, alkenyl, nitro, amino, alkoxyl, amido, and the like.

“MRL-871” as used in the present application means a selective inhibitor (4-[1[2-Chloro-6-(trifluoromethyl)benzoyl]-1H-indazol-3-yl]-benzoic acid; of ROR-γt via allosteric inverse agonism (see also Scheepstra et al. Nat Commun. 2015; 6: 8833). MRL-871 has the following chemical structure:

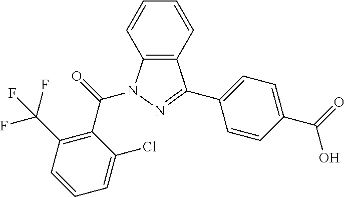 [SMILES: O=C(O)c1ccc(-c2nn(C(=O)c3c(Cl)cccc3C(F)(F)F)c3ccccc23)cc1]

“Potency” as used in the present application refers to a measure of a compound's activity expressed in terms of the amount required to produce an effect of given intensity. It is understood that if potency of a compound is increased, then the amount of a compound needed to achieve the desired effect is decreased. Conversely, if the potency of a compound is decreased, then the amount of this compound needed to achieve the desired effect is increased.

### DETAILED DESCRIPTION OF THE INVENTION

In a first aspect the application relates to a compound having formula (I):

(I)

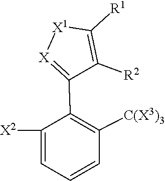

or salts or solvates thereof;

wherein:
X and X¹ are independently N or O;
X² and X³ are each independently an H or a halogen, wherein preferably X² is Cl and X³ is F;
R¹ is a hydrocarbyl, preferably alkyl or aryl, wherein said alkyl and aryl may be unsubstituted or substituted, wherein said aryl and alkyl may comprise one or more heteroatoms, wherein said alkyl may be linear, branched or cyclic; and
R² is

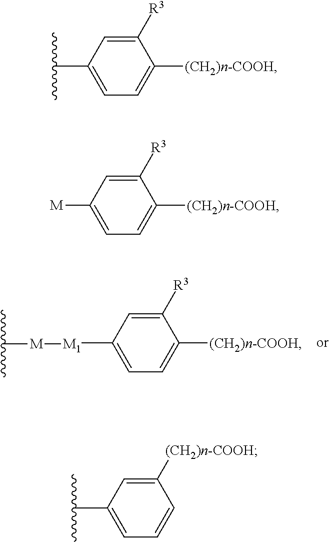

wherein
R³ is a halogen or H, wherein preferably the halogen is F;
n is 0 or an integer equal or greater than 1; and
M and M₁ are, each independently selected from the group consisting of —NH—, —CH₂—, —SO₂, —NH—CH₂—, —COH, —O—, ═N—, and —C(O).

The compounds according to the invention are substituted cyclic alkyl compounds comprising one or more heteroatoms or substituted heterocycles and it is believed the compounds according to the invention act as inhibitors of ROR-yt by targeting the allosteric site of ROR-yt. These compounds can therefore be used in the prophylaxis or treatment of various (auto)immune diseases and allergic disorders associated with the receptor ROR-γt (retinoid acid-related orphan receptor γt) and/or its role in the regulation of helper T-17 (Th 17) cell differentiation. The compounds according to the invention can be used as inhibitors, possibly via an allosteric inverse agonist mechanism, of (human) ROR-γt.

In some embodiments, the type of salt may be any type and non-limiting examples of salts of a compound having formula (I) may be selected from the non-limitative group consisting of metal salts, ammonium salts, salts with organic base, salts with inorganic acid, salts with organic acid, and salts with basic or acidic amino acids.

In some embodiments the metal salts may be selected from the non-limitative group consisting of alkaline metal salts, including sodium salt, potassium salt; alkaline earth metal salts including calcium salt, magnesium salt, barium salt; and aluminum salts.

In some embodiments, the salt with organic base may be selected from the non-limitative group consisting of salts with trimethylamine, triethylamine, pyridine, picoline, 2,6-lutidine, ethanolamine, diethanolamine, triethanolamine, cyclohexylamine, dicyclohexylamine, and N, N′-dibenzylethylenediamine.

In some embodiments, the salt with inorganic acid may be selected from the non-limitative group consisting of salts with hydrochloric acid, hydrobromic acid, nitric acid, sulfuric acid, and phosphoric acid.

In some embodiments, the salt with organic acid may be selected from the non-limitative group consisting of salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, and p-toluenesulfonic acid.

In some embodiments, the salt with basic amino acid may be selected from the non-limitative group consisting of salts with arginine, lysine, and ornithine. Preferable examples of the salt with acidic amino acid include salt with aspartic acid, glutamic acid and the like.

In a preferred embodiment the compound having formula (I) is a trisubstituted isoxazole, in which X is N and X¹ is O. In another preferred embodiment there is provided for a compound according to formula (I) wherein X is N an X¹ is O, and wherein X² is CL and X³ is F, and wherein R¹, R², and R³ are as described above.

The current inventors have surprisingly seen that the nature of R¹ and/or R² in the compounds having formula (I) influences the potency of the compound; which means that the amount of a compound having formula (I) needed to achieve a ROR-γt inhibitory effect is influenced. In the present invention it will be understood that a positive or beneficial effect on the potency of a compounds means that the amount of a compound needed to achieve a ROR-γt inhibitory effect is reduced as compared to compounds not having such a positive or beneficial effect on the potency. In this sense, the compounds according to the invention have a half-maximum inhibitory concentration (IC₅₀) of less than 100 μM.

For example, a compound according to the invention, in which R¹ is selected from the group consisting of a substituted or unsubstituted aryl and a substituted or unsubstituted cyclic alkyl comprising one or more heteroatoms, may provide for a higher potency than a compound, not according to the invention, in which R¹ is an alkyl group, such as methyl.

In an embodiment the substituted or unsubstituted aryl is a substituent derived from a substituted or unsubstituted polycyclic aromatic, for example, naphthyl, preferably 1-naphthyl.

In an embodiment the unsubstituted aryl is phenyl. In another embodiment the substituted aryl is phenoxyl, preferably 3-phenoxyl.

In an embodiment the substituted or unsubstituted cyclic alkyl comprising one or more heteroatoms compound is selected from the group consisting of pyrrolyl, furyl, and thienyl; preferably selected from the group consisting of 3-pyrrolyl, 2-furyl, and 2-thienyl.

The current inventors have also observed that the inclusion of a single methylene unit between the amide and benzoic acid moieties, i.e. when in R², M₁ is —NH—CH₂— and n is zero, can provide for a beneficial effect on the potency of the compound.

In addition, a single carbon-carbon bond between the isoxazole and the benzoic acid moiety, i.e. when R² is

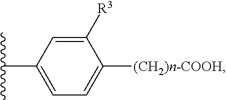

R³ is H and n is zero, can be provide for a beneficial effect on the potency of the compound.

In an embodiment, M and M₁ are, each independently selected from the group consisting of —NH—, —CH₂—, —SO₂, —NH—CH₂—, —COH, —O—, ═N— and —C(O). In a preferred embodiment, M and M1 are, each independently selected from the group consisting of —NH—, —CH₂—, —SO₂, —NH—CH₂—, —COH, —O—, and —C(O).

Examples of compounds according to the invention and having formula (I) are the following:

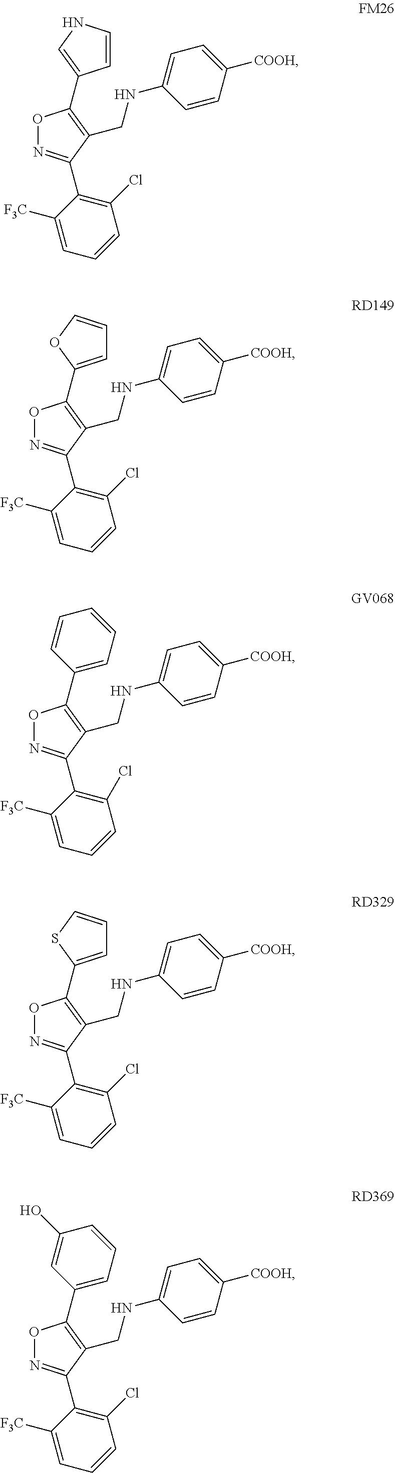 [SMILES: O=C(O)c1ccc(NCc2c(-c3c(Cl)cccc3C(F)(F)F)noc2-c2cc[nH]c2)cc1 ; O=C(O)c1ccc(NCc2c(-c3c(Cl)cccc3C(F)(F)F)noc2-c2ccco2)cc1 ; O=C(O)c1ccc(NCc2c(-c3c(Cl)cccc3C(F)(F)F)noc2-c2ccccc2)cc1 ; O=C(O)c1ccc(NCc2c(-c3c(Cl)cccc3C(F)(F)F)noc2-c2cccs2)cc1 ; O=C(O)c1ccc(NCc2c(-c3c(Cl)cccc3C(F)(F)F)noc2-c2cccc(O)c2)cc1]

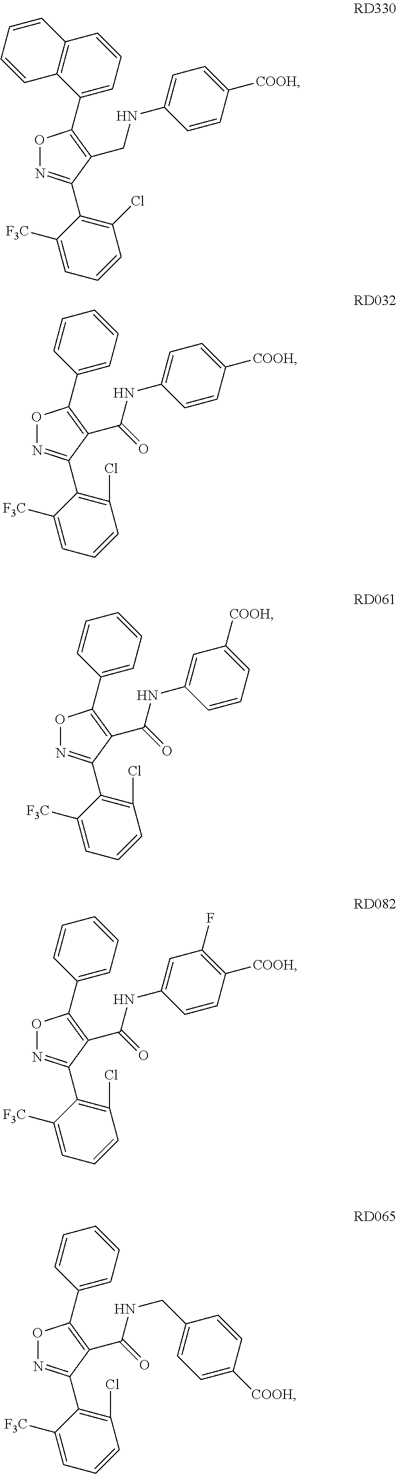

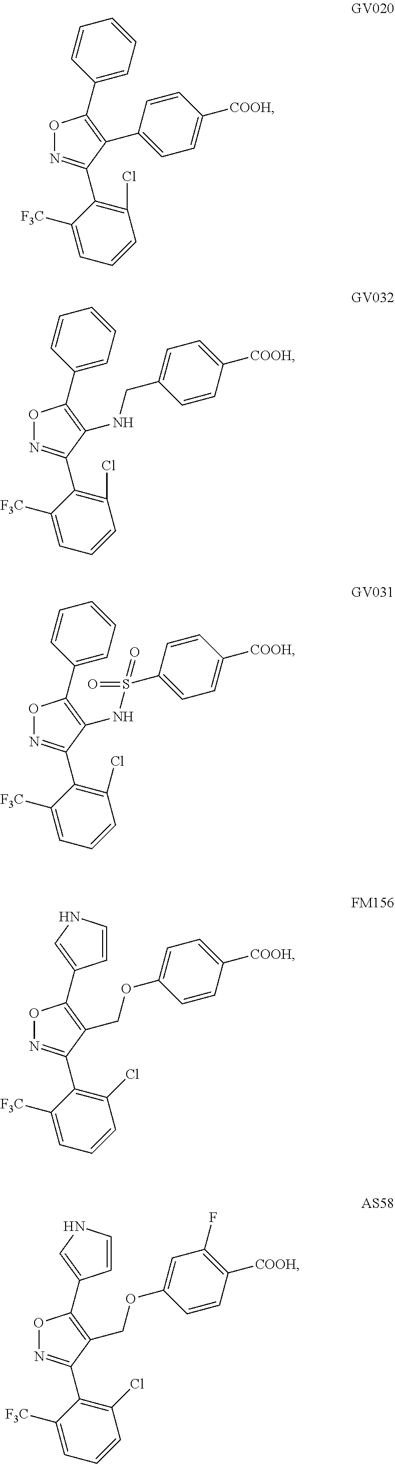

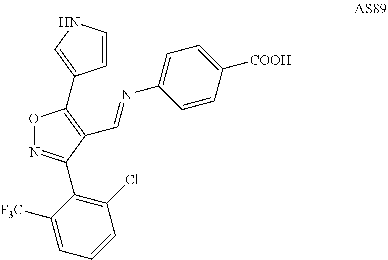 [SMILES: O=C(O)c1ccc(/N=C/c2c(-c3c(Cl)cccc3C(F)(F)F)noc2-c2cc[nH]c2)cc1]

or a salt or a solvate thereof.

In a second aspect, the invention relates to a composition comprising a compound represented by formula (I) according to the first aspect of the invention, or a salt or solvate thereof, and preferably at least one carrier.

In a third aspect, the invention relates to a pharmaceutical composition comprising a compound represented by formula (I) according to the first aspect of the invention, or a pharmaceutically acceptable salt or solvate thereof, and preferably at least one pharmaceutically acceptable carrier.

The term “excipient” and “carrier” may be used interchangeably. The term “composition”, as for example used in “pharmaceutical composition”, is intended to encompass a product comprising the active ingredient(s) as disclosed herein, and, preferably at least one additional (inert) compound or ingredient (for example one or more pharmaceutically-acceptable excipient) that make up the carrier. Accordingly, the pharmaceutical compositions of the present invention encompass any composition made by admixing a compound of Formula I, optionally additional active ingredient(s), and (pharmaceutically acceptable) excipients.

The pharmaceutical compositions of the present invention comprise a compound represented by Formula I, or a pharmaceutically acceptable salt or solvate thereof, as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy; preferably the pharmaceutical composition is presented in dosages for oral intake.

The compound can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. It can be administered alone or with a pharmaceutical carrier selected on the basis of the chosen route of administration and standard pharmaceutical practice.

A suitable dosage of the compound according to the invention can be established by the skilled person by performing dose finding studies. A dosage and/or dosage regimen for the compound according to the invention, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

In a fourth aspect, the invention relates to a compound represented by formula (I) according to the first aspect of the invention, or a pharmaceutically acceptable salt or solvate thereof, for use as a medicament.

The exact dose and regimen of administration of the active ingredient, or a pharmaceutical composition thereof, may vary with the particular compound, the route of administration, and the age and condition of the individual subject to whom the medicament is to be administered.

The compound according to the invention may, for example, be administered in a single daily dose, or the total daily dosage may be administered in divided doses of two, three, or four times daily.

The compound according to the invention may be administered in admixture with suitable pharmaceutical diluents, excipients, or carriers selected with respect to the intended form of administration.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulphate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and colouring agents can also be incorporated into the mixture. Suitable binders include starch, gelatine, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Suitable pharmaceutical carriers are described in Remington's Pharmaceutical Sciences, Mack Publishing Company, a standard reference text in this field.

In a fifth aspect, the invention relates to a compound represented by formula (I) according to the first aspect of the invention, or a pharmaceutically acceptable salt or solvate thereof, for use in the prophylaxis or treatment of an autoimmune disease or allergic disorder in a subject. In an embodiment, the autoimmune disease or allergic disorder is selected from psoriasis, rheumatoid arthritis, systemic lupus erythromatosis, scleroderma, Type II diabetes, asthma, allergic rhinitis, allergic eczema, multiple sclerosis, juvenile rheumatoid arthritis, juvenile idiopathic arthritis, inflammatory bowel diseases, ulcerative colitis, Crohn's disease, graft versus host disease, spondyloarthropathies and uveitis.

In a sixth aspect, the invention relates to a compound represented by formula (I) according to the first aspect of the invention for use in a method for the prophylaxis or treatment of an autoimmune disease or allergic disorder in a subject wherein the method comprises administering to the subject a therapeutically effective amount of a compound represented by formula (I) according to the first aspect of the invention, or a pharmaceutically acceptable salt or solvate thereof.

In an embodiment, the method comprises administering to the subject a compound represented by formula (I) according to the first aspect of the invention, or a (pharmaceutically) acceptable salt or solvate thereof.

In a seventh aspect, the invention relates to a compound represented by formula (I) according to the first aspect of the invention, or a pharmaceutically acceptable salt or solvate thereof, for use in a method for inhibiting ROR-yt in a subject wherein the method comprises administering to the subject a compound represented by formula (I) according to the first aspect of the invention, or a (pharmaceutically) acceptable salt or solvate thereof.

It will be understood that “a therapeutically effective amount of a compound represented by formula (I)”, refers to an amount sufficient to achieve the intended purpose; e.g. to prevent or to treat an autoimmune disease or allergic disorder and/or to inhibit ROR-yt in a subject, preferably a human subject.

In an eight aspect, the invention relates to a use of a compound represented by formula (I) according to the first aspect of the invention, or a (pharmaceutically) acceptable salt or solvate thereof, for the production of a pharmaceutical composition.

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

It will be understood that all details, embodiments and preferences discussed with respect to one aspect of embodiment of the invention is likewise applicable to any other aspect or embodiment of the invention and that there is therefore not need to detail all such details, embodiments and preferences for all aspect separately./pct

Having now generally described the invention, the same will be more readily understood through reference to the following examples which is provided by way of illustration and is not intended to be limiting of the present invention. Further aspects and embodiments will be apparent to those skilled in the art.

Method of Preparation

The compounds, described herein, according to general formula (I) are prepared using techniques known to one skilled in the art of organic synthesis; this means that the person skilled in the art of organic synthesis will be able to prepare the compounds according to the general formula (I). An example is given when considering the preparation of a compound according to formula (I), in which R² is

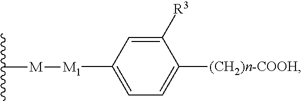

in which M is —C(O), M₁ is —NH—, R³ is H and n is zero; this compound was prepared following scheme 1.

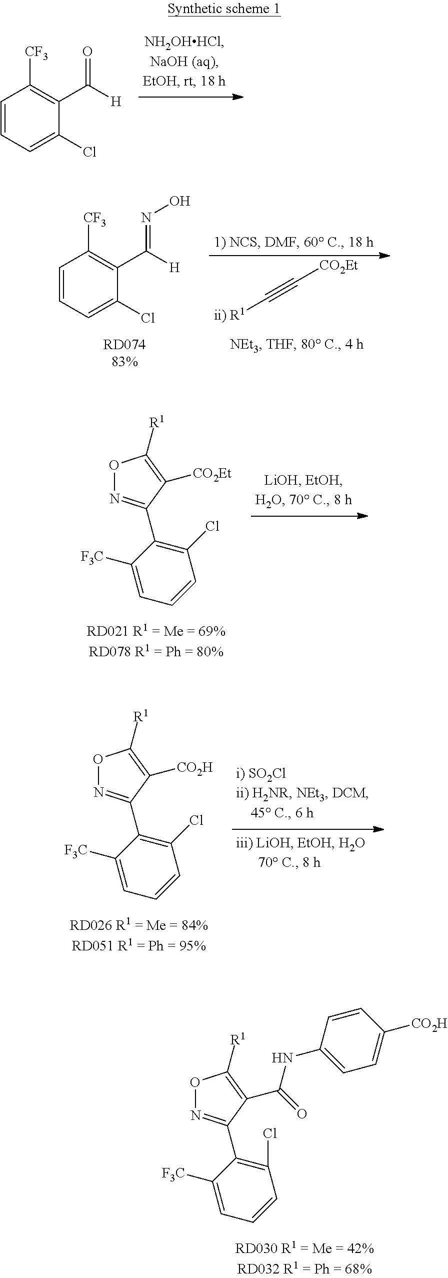

The compound prepared according to scheme 1 and having formula (I) was synthesized via a 3+2 dipolar cycloaddition of a nitrile oxide and a commercially available alkyne. The regiochemistry of the resulting trisubstituted isoxazole esters and products was confirmed by 2D-NMR experiments. Ester hydrolysis followed by amide coupling of tert-butyl-4-amino benzoate via the respective acid chloride and finally deprotection of the tent-butyl ester furnished the target compounds RD030 and RD032 in an efficient manner.

It is understood that, where specific acids, bases, reagents, coupling agents, solvents, etc. are mentioned, it is understood that other suitable acids, bases, reagents, coupling agents, solvents etc. may be used and are included within the scope of the present invention. The modifications to reaction conditions, for example, temperature, duration of the reaction or combinations thereof, are envisioned as part of the present invention. The compounds obtained using the general reaction sequences may be of insufficient purity. These compounds can be purified using any of the methods for purification of organic compounds known to persons skilled in the art, for example, crystallization or silica gel or alumina column chromatography using different solvents in suitable ratios. All possible geometrical isomers and stereoisomers are envisioned within the scope of this invention.

The starting materials used herein are commercially available or were prepared by methods known in the art to those of ordinary skill or by methods disclosed herein.

### EXAMPLES

General introduction

Experiments were performed in order to show the allosteric mode-of-action of the compounds according to the invention.

TR-FRET

A first experiment performed was time-resolved Fluorescence Resonance Energy Transfer (TR-FRET) coactivator recruitment assay (Scheepstra et al.Nat Commun. 2015; 6: 8833 and Stafslien D. et al. Mol Cell Endocrinol 2007; 1-2; 82-89). The coactivator recruitment TR-FRET assay was performed in the presence of increasing fixed concentrations of cholesterol, a well characterized orthosteric agonist. Ligands acting via the orthosteric site will then compete for binding with cholesterol and the observed IC₅₀'s increases as the cholesterol concentration increases. Allosteric inverse antagonists, as the compounds according to the invention, do not compete for binding.

Ligand Displacement Assay

A second experiment was an allosteric ligand displacement assay (Scheepstra et al.Nat Commun. 2015; 6: 8833). In such assay, allosteric inverse agonists, as the compounds according to the invention, will displace an allosteric probe with high potency. Orthosteric inverse agonists only show a weak effect.

Materials and Methods

The activity data of a compound according to the invention was compared with the activity data of two known inhibitors of ROR-γt via allosteric inverse agonism and one inhibitor acting via the orthosteric site. The compounds used are shown in Table 1

TABLE 1

| Compound | Structure |
|---|---|
| 1 | FM26 |
| | 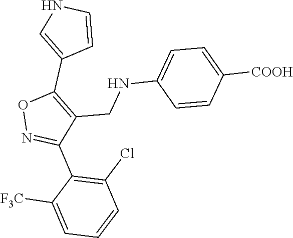 |
| A | 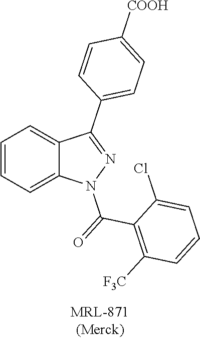 |
| B | 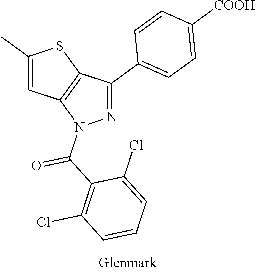 |
| C | 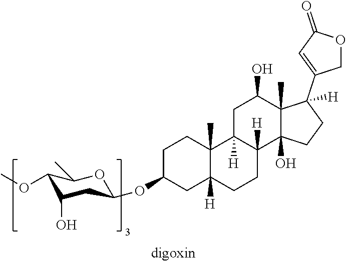 |

In Table 1, compound 1 is a compound according to the invention whilst Compounds A, B and C are compounds not according to the invention.

TR-FRET

The primary assay to demonstrate biological activity is a time-resolved FRET (TR-FRET) coactivator recruitment assay. Assays were conducted using 100 nM N-terminal biotinylated SRC-1 box2 peptide and 20 nM His6-RORyt ligand binding domain in buffer containing 10 mM 4-(2-hydroxyethyl)-1-piperazineethanesulfonic acid (HEPES—pH of 7.5), 150 mM NaCl, 5 mM Dithiothreitol (DTT), 0.1% Bis(trimethylsilyl)acetamide (BSA) (w/v) and 0.1 mM 3-[(3-Cholamidopropyl)dimethylammonio]-1-propanesulfonate hydrate (CHAPs). A terbium labelled anti-His antibody (CisBio Bioassays, 61HISTLA) and D2-labelled streptavidin (CisBio Bioassays, 610SADLA) were used at the concentrations recommended by the supplier. Compounds (dissolved in Dimethyl sulfoxide—DMSO) were titrated using a 2× dilution series from a highest final concentration of 100 pM such that the final DMSO concentration was 2% v/v throughout. The plate was incubated at room temperature for 30 min and centrifuged before reading on a Tecan infinite F500 plate reader using the parameters recommended by CisBio Bioassays (Scheepstra et al.Nat Commun. 2015; 6: 8833)

Ligand Displacement Assay

To provide further evidence for allosteric ligand binding we used an orthogonal assay to directly probe for allosteric ligand binding (as opposed to measuring the indirect effects on coactivator recruitment). In this assay compounds were labelled with AlexaFluor 647 and FRET was measured in the presence of His6-RORyt and an anti-His terbium cryptate antibody. The Alexa Fluor 647 is a bright, far-red-fluorescent dye with excitation ideally suited for the 594 nm or 633 nm laser lines. For stable signal generation in imaging and flow cytometry, Alexa Fluor 647 dye is pH-insensitive over a wide molar range.

Results

TR-FRET

In the presence of fixed concentrations of an orthosteric agonist (cholesterol), compound C competed for the same binding site, as it shows a cholesterol-dependent activity profile. Compound C is an orthosteric inverse agonist whereby cholesterol concentration-dependent IC₅₀ values were observed. Conversely, the compounds 1, A and B effectively perturbed coactivator recruitment in a manner that was independent of increasing concentrations of cholesterol. In these cases, and in particular for compound 1, the present inventors also observed that IC₅₀ values actually decreased and hill slopes steepened.

TABLE 1

| | 0 μM Cholesterol | | 0.1 μM Cholesterol | | 1.0 μM Cholesterol | |
|---|---|---|---|---|---|---|
| Compound | IC₅₀ (nM) | Hill slope | IC₅₀ (nM) | Hill slope | IC₅₀ (nM) | Hill slope |
| 1 | 98.8 ± 12.4 | −0.45 ± 0.02 | 9.9 ± 0.6 | −0.74 ± 0.03 | 11.3 ± 0.5 | −0.86 ± 0.03 |
| B | 260.8 ± 22.4 | −0.59 ± 0.03 | 39.9 ± 1.9 | −0.65 ± 0.02 | 34.5 ± 1.2 | −0.76 ± 0.02 |
| A | 75.7 ± 4.1 | −0.69 ± 0.02 | 29.1 ± 10.6 | −0.83 ± 0.02 | 24.8 ± 0.7 | −1.01 ± 0.03 |
| C | 774.1 ± 81.6 | −0.53 ± 0.03 | 5474 ± 456 | −0.54 ± 0.02 | 32366 ± 1694 | −0.87 ± 0.04 |

Table 1 shows the values of IC₅₀ for different cholesterol concentrations. These results not only provide strong evidence for binding to the allosteric pocket, but also indicate that this is not necessarily independent of orthosteric site occupancy.

The values of IC₅₀ for different compounds according to the invention were also determined for a cholesterol concentration of 0 μM and with the TR-FRET assay. These results are shown in table 2.

TABLE 2

| | |
|---|---|
| FM26 | IC₅₀ = 264 ± 23 nM |
| 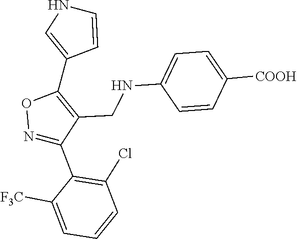 | |
| RD149 | IC₅₀ = 1.1 ± 0.1 μM |
| 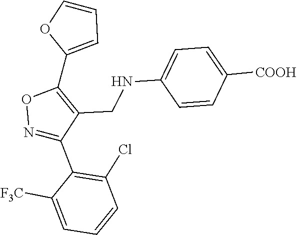 | |
| GV068 | IC₅₀ = 9.6 ± 0.6 μM |
| 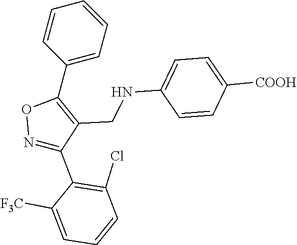 | |
| RD329 | IC₅₀ = 1.8 ± 0.2 μM |
| 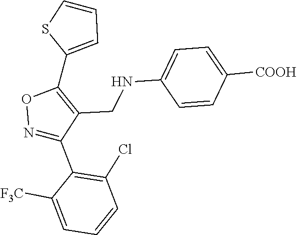 | |
| RD369 | IC₅₀ = 6.6 ± 0.5 μM |
| 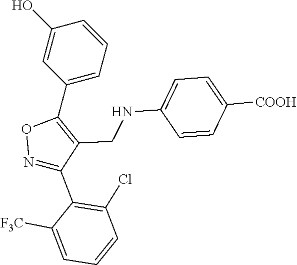 | |
| RD330 | IC₅₀ = n.d. |
| 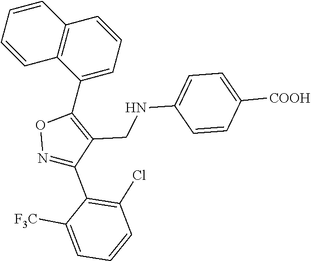 | |
| RD032 | IC₅₀ = 53.5 ± 2.9 μM |
| 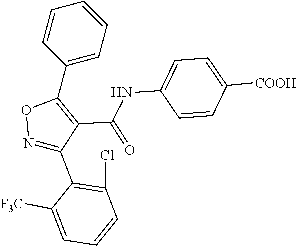 | |
| RD061 | IC₅₀ = 73.9 ± 3.4 μM |
| 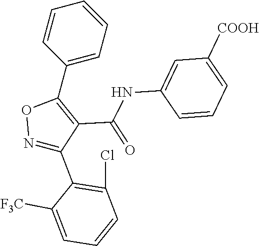 | |
| RD082 | IC₅₀ = 91.1 ± 4.6 μM |
| 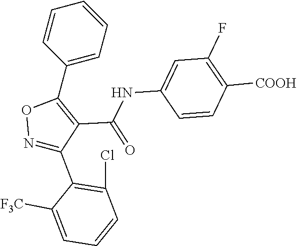 | |
| RD065 | IC₅₀ = 8.8 ± 0.5 μM |
| 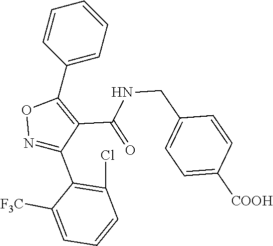 | |
| GV020 | IC₅₀ = 8.2 ± 0.3 μM |
| 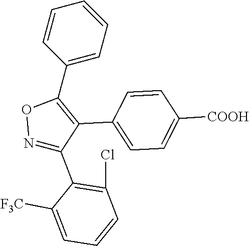 | |
| GV032 | IC₅₀ = 30.9 ± 1.3 μM |
| 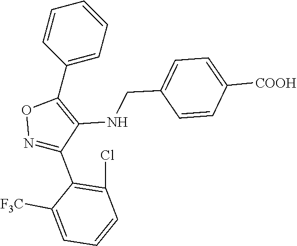 | |
| GV031 | IC₅₀ = 62.6 ± 4.4 μM |
| 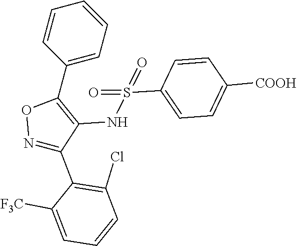 | |
| FM156 | IC₅₀ = 36 ± 4 nM |
| 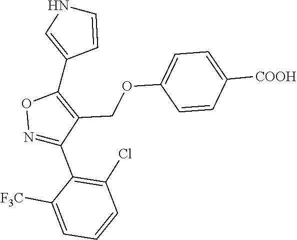 | |
| AS58 | IC₅₀ = 5.2 ± 0.6 μM |
| 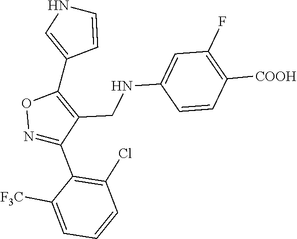 | |
| AS89 | IC₅₀ = 46.6 ± 5.2 μM |
| 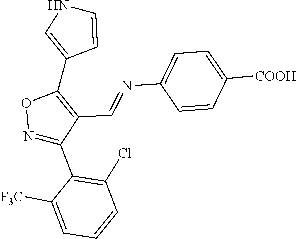 | |

Ligand Displacement Assay

To provide further evidence for allosteric ligand binding we used an orthogonal assay to directly probe for allosteric ligand binding (as opposed to measuring the indirect effects on coactivator recruitment). Compound A was shown to compete for binding and displaced the Alexa Fluor 647 probe (Scheepstra et al. Nat Commun. 2015; 6: 8833) with an IC₅₀=40.2±0.3 nM. This was also the case for compound 1 according to the invention, for which IC₅₀=64.4±8.7 nM and for compound B, for which IC₅₀=117.3±12.1 nM. This behavior was not the case for the orthosteric inverse agonist, i.e. compound C, which showed displacement of the Alexa Fluor 647 probe only at concentrations IC₅₀>50 μM. These results corroborated those from the competitive coactivator recruitment assay and provide strong evidence for allosteric binding of the compound 1 according to the invention. They also provide another indication that allosteric binding is not entirely independent of orthosteric binding site occupancy.

| | AlexaFluor Probe | Coactivator |
|---|---|---|
| Compound | Competition IC₅₀ (nM) | Recruitment IC₅₀ (nM) |
| 1 | 64.4 ± 8.7 | 98.8 ± 12.4 |
| B | 117.3 ± 12.1 | 260.8 ± 22.4 |
| A | 40.2 ± 0.3 | 75.7 ± 4.1 |
| C | >5000 | 774.1 ± 81.6 |

Having now fully described this invention, it will be appreciated by those skilled in the art that the same can be performed within a wide range of equivalent parameters, concentrations, and conditions without departing from the spirit and scope of the invention and without undue experimentation.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art. The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

1. A composition comprising a compound having formula (I): 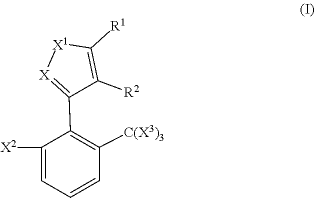
or salts or solvates thereof;
wherein:
X and X¹ are independently N or O;
X² and X³ are each independently an H or a halogen, wherein preferably X² is Cl and X³ is F;
R¹ is a hydrocarbyl, preferably alkyl or aryl, wherein said alkyl and aryl may be unsubstituted or substituted, wherein said aryl and alkyl may comprise one or more heteroatoms, wherein said alkyl may be linear, branched or cyclic; and
R² is 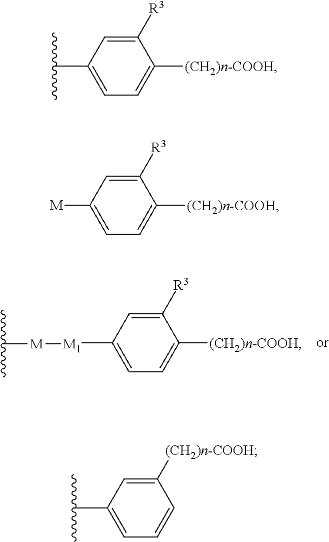
wherein
R³ is a halogen or H, wherein preferably the halogen is F;
n is 0 or an integer equal or greater than 1; and
M and M₁ are, each independently selected from the group consisting of —NH—, —CH₂—, —SO₂, —NH—CH₂—, —COH, —O—, ═N— and —C(O).

2. The composition of claim 1, wherein said compound is selected from the group consisting of 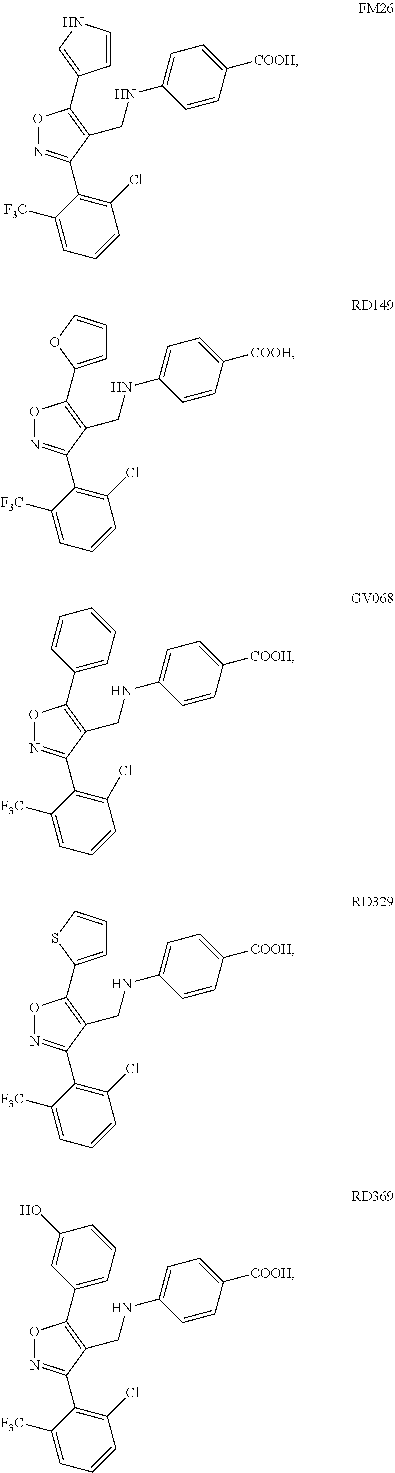 [SMILES: O=C(O)c1ccc(NCc2c(-c3c(Cl)cccc3C(F)(F)F)noc2-c2cc[nH]c2)cc1 ; O=C(O)c1ccc(NCc2c(-c3c(Cl)cccc3C(F)(F)F)noc2-c2ccco2)cc1 ; O=C(O)c1ccc(NCc2c(-c3c(Cl)cccc3C(F)(F)F)noc2-c2ccccc2)cc1 ; O=C(O)c1ccc(NCc2c(-c3c(Cl)cccc3C(F)(F)F)noc2-c2cccs2)cc1 ; O=C(O)c1ccc(NCc2c(-c3c(Cl)cccc3C(F)(F)F)noc2-c2cccc(O)c2)cc1] 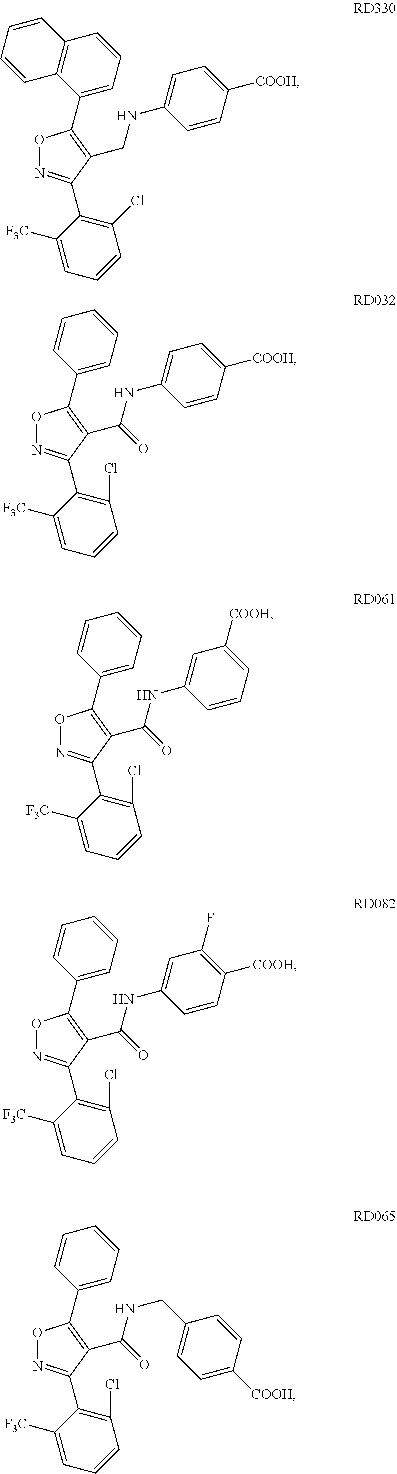 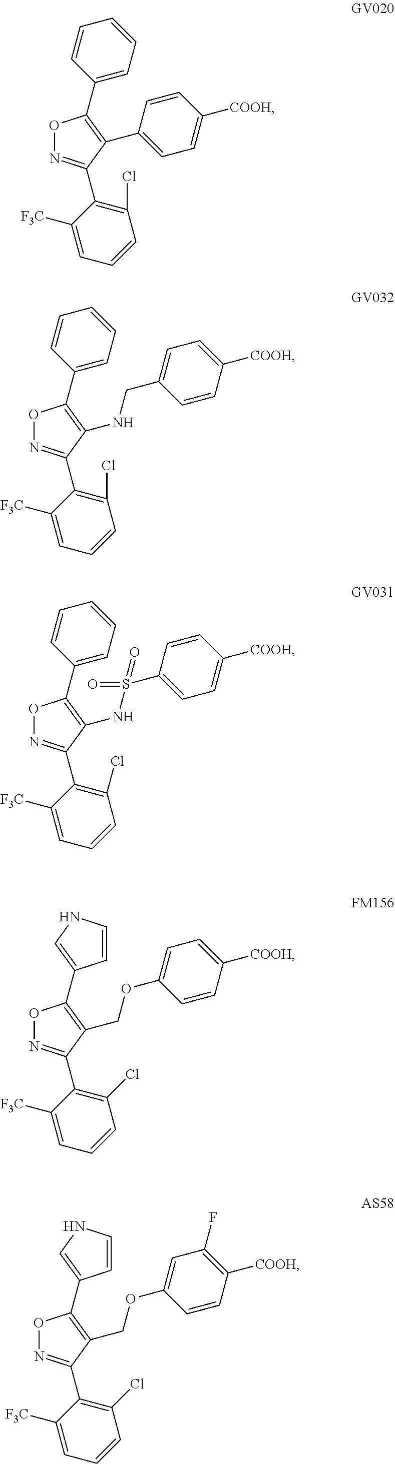 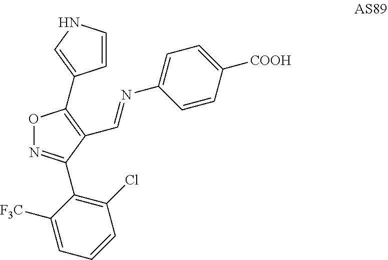 [SMILES: O=C(O)c1ccc(/N=C/c2c(-c3c(Cl)cccc3C(F)(F)F)noc2-c2cc[nH]c2)cc1]
or a salt or a solvate thereof.

3. The composition of claim 1, wherein the composition further comprises at least one carrier.

4. The composition of claim 1, wherein the composition is a pharmaceutical composition, wherein the composition further comprises at least one pharmaceutically acceptable carrier.

5. The composition of claim 1, wherein the composition is configured for use as a medicament.

6. The composition of claim 1, wherein the composition is configured for use in the prophylaxis or treatment of an autoimmune disease or allergic disorder in a subject.

7. The composition of claim 6, wherein the autoimmune disease or allergic disorder is selected from psoriasis, rheumatoid arthritis, systemic lupus erythromatosis, scleroderma, Type II diabetes, asthma, allergic rhinitis, allergic eczema, multiple sclerosis, juvenile rheumatoid arthritis, juvenile idiopathic arthritis, inflammatory bowel diseases, ulcerative colitis, Crohn's disease, graft versus host disease, spondyloarthropathies and uveitis.

8. A method for the prophylaxis or treatment of an autoimmune disease or allergic disorder in a subject and/or for inhibiting ROR-yt in a subject, wherein the method comprises administering to the subject a therapeutically effective amount of a compound represented by formula (I) according to claim 1, or a pharmaceutically acceptable salt or solvate thereof.

9. (canceled)

10. Use of a compound represented by formula (I) according to claim 1, or a (pharmaceutically) acceptable salt or solvate thereof, for the production of a pharmaceutical composition.
